(19) United States
(12) Patent Application Publication
(10) Pub. No.: US 2005/0013871 A1
(43) Pub. Date: Jan. 20, 2005

(54) Pharmaceutical composition for the treatment of itch

(76) Inventors: Sarfaraz Niazi, Deerfield, IL (US); Anjum Niazi, Deerfield, IL (US)

Correspondence Address: SARFARAZ K. NIAZI, 20 RIVERSIDE DRIVE, DEERFIELD, IL, 60015, US

(21) Appl. No.: 10/604,424

(22) Filed: Jul. 20, 2003

(51) Int. Cl. A61K 35/64; A61K 35/78

(52) U.S. Cl. 424/539; 424/767; 424/769

(57) ABSTRACT

Disclosed is a topical preparation for the treatment of topical itch in humans and animals. The said composition consists of Opuntia, Propolis, Stearic Acid, Beeswax, Vegetable Oil and β-sitosterol. Itch includes scratch reaction itch, anal itch, or irritant itch due to plants (e.g., poison ivy), insect bite, sunburn, chemical itch, eczema, pruritis dermatitis, diabetic skin itch, aging skin itch, foot-itch, chickenpox, jock itch, hives, itch of healing burns and wounds, dry winter skin itch, and stress-related scalp itch, etc

### BACKGROUND OF INVENTION

Itch is a well known sensory state associated with the desire to scratch. As with pain, itch can be produced by a variety of chemical, mechanical, thermal or electrical stimuli. In addition to the difference in the sensory quality of itch and pain, they also differ in that (1) itch, unlike pain, can only be evoked from the superficial layers of skin, mucosa, and conjunctiva, and (2) itch and pain usually do not occur simultaneously from the same skin region; in fact, mildly painful stimuli, such as scratching, are effective in eliminating itch. In addition, the application of histamine to skin produces itch but not pain. Itch and pain are further dissociated pharmacologically: itch appears to be insensitive to opiate and non-steroidal anti-inflammatory drug (NSAID) treatment, both of which are effective in treating pain.

Although itch and pain are of a class in that both are modalities of nociception transmitted by small unmyelinated C fibers, evidence that itch is not just a variety of low threshold pain is overwhelming. Itch leads to the reflex or urge to scratch; pain leads to withdrawal. Removal of the epidermis eliminates itch but causes pain. Analgesics, particularly opiods, relieve pain but often cause itch (see, for example J. Am. Acad. Derm. 24: 309-310, 1991). There can be no doubt that itching is of eminent clinical importance; many systemic and skin diseases are accompanied by persistent or recurrent itch attacks. Current knowledge suggests that itch has several features in common with pain but exhibits intriguing differences as well (see, for example, W. Mager, IASP Newsletter, pp. 4-7, September/October 1996).

Experimental focal itch stimuli are surrounded by a halo of seemingly unaffected tissue where light tactile stimuli are capable of eliciting itch-like sensations. The term itchy skin or allokinesis has been coined for these secondary sensations that are reminiscent of the features of secondary hyperalgesia evolving around a painful focus. A crucial observation is that itch and pain usually do not coexist in the same skin region and a mild noxious stimulus such as scratching is in fact the singly most effective way to abolish itch. This abolition of itch can be prolonged producing an ‘antipruritic state’. Although mild scratch is often not painful, microneurographic recordings from humans have directly determined that such stimuli are among the most effective ways to excite cutaneous unmyelinated nociceptive afferents. (See, for example: Shelly, W. B. and Arthur, R. P. (1957) Arch. Dermatol. 76, 296-323; Simone, D. A. et al. (1987) Somatosens. Res. 5, 81-92; Graham, D. T., Goodell, H. and Wolff, H. G. (1951) J. Clin. Invest. 30, 37-49; Simone, D. A., Alreja, M. and LaMotte, R. H. (1991) Somatosens, Mot. Res. 8, 271-279; Torebjork, E (1985) Philos. Trans. R. Soc. London Ser. B 308, 227-234; and Valbo, A. B., Hagbarth, K. E., Torebjork, H. E. and Wallin, B. G. (1979) Physiol. Rev. 59, 919-957).

Physiologically, there is evidence that substance P released from nociceptor terminals can cause the release of histamine from mast cells. Activation of mast cells, with release of the pruritogen histamine, occurs in immediate type hypersensitivity diseases, such as anaphylactic reactions and urticaria. Urticarial eruptions are distinctly pruritic and can involve any portion of the body, and have a variety of causes beyond hypersensitivity, including physical stimuli such as cold, solar radiation, exercise and mechanical irritation. Other causes of prutitus include: chiggers, the larval form of which secretes substance that creates a red papule that itches intensely; secondary hyperparathyroidism associated with chronic renal failure; cutaneous larva migrans, caused by burrowing larvae of animal hookworms; dermal myiasis, caused by maggots of the horse botfly, which can afflict horseback riders; onchocerciasis (“river blindness”) caused by filarial nematodes; pediculosis, caused by lice infestations; enterobiasis (pinworm) infestations, which afflict about 40 million Americans, particularly school children; schistosome dermatitis (swimmer's itch); psoriasis; poison ivy and asteatotic eczema (“winter itch”). The role of histamine or other endogenous pruritogens in mediating itch associated with these and other pruritic conditions, such as atopic dermatitis, its not yet well established. For atopic dermatitis, in particular, it appears that itch is not inhibited by antihistamines, but by cyclosporin A, a drug which inhibits the production of cytokines which have been proposed as potential pruritogens.

Current therapies for the treatment of itch include a variety of topical and systemic agents, such as steroids, antihistamines, and some psychotherapeutic tricyclic compounds, such as doxepin hydrochloride. The limitations of these agents are well known to medical practitioners, and are summarized in the “Warnings” and “Precautions” sections for the individual agents listed in PDR Generics. In particular, the lack of complete efficacy of antihistamines is well known, but antihistamines are frequently used in dermatology to treat prutitus due to urticaria, atopic dermatitis, contact dermatitis, psoriasis, and a variety of other conditions. Although sedation has been a frequent side effect of conventional systemically administered antihistamines, a new generation of antihistamines has been developed that are nonsedating, apparently due to their inability to cross the blood-brain barrier.

Scratch reaction itch, anal itch, irritant itch are due to plants (e.g., poison ivy), insect bite, sunburn, chemical itch, eczema, pruritis dermatitis, diabetic skin itch, aging skin itch, foot-itch, chickenpox, jock itch, hives, itch of healing burns and wounds, dry winter skin itch, and stress-related scalp itch, etc.

Atopic dermatitis is a skin inflammation that usually develops in early childhood and continues into the teen years. While few adults who had it during childhood still have severe itch and rash, many continue to have milder symptoms and “sensitive” skin problems. Antihistamines reduce itching by blocking chemical (histamine) processes in an allergic reaction. For many years, antihistamines have been used to reduce the itch caused by atopic dermatitis. Sedating antihistamines have been favored for treating children who cannot sleep because of severe itch at night. However, recent research suggests that atopic dermatitis itch is not caused by histamines. (Kristal L, Klein P (2000). Atopic dermatitis in infants and children. Pediatric Clinics of North America, 47(4): 877895; Klein P, Clark R (1999). An evidence-based review of the efficacy of antihistamines in relieving pruritus in atopic dermatitis. Archives of Dermatology, 135: 15221525.).

Pruritus is the medical word for itch. It is defined as a sensation that provokes the desire to scratch. Itching can be a significant source of frustration and discomfort for patients. When severe, it can lead to loss of sleep, anxiety, and depression. The exact cause of an itch is unknown and is a complex process. Ultimately it involves nerves in the skin responding to certain chemicals such as histamine, and then processing these signals in the brain. Pruritus can be a symptom of certain skin diseases, and sometimes a manifestation of an internal process. In other patients where there is no evidence of skin or internal disease, pruritus may be due to faulty processing of the itch sensation within the nervous system.

There are many skin diseases that may have itching associated with a rash as a prominent symptom. Examples would be hives, chicken pox, and eczema. Some skin conditions only have symptoms of pruritus without having an apparent rash. Dry skin, for example, is very common in the elderly, and can really itch (especially in the winter), with no visual signs of a rash. Pruritus is usually secondary to subtle dry skin, but it may be a manifestation of an internal condition. Some parasitic infestations of the skin, such as scabies and lice, may be very itchy. Often the dermatologist will be able to diagnose these conditions by examining the skin. When pigmented moles itch, a dermatological opinion should be sought to exclude a malignant change in the mole. Sometimes, however, a skin scraping or a biopsy may be needed to help make the diagnosis.

There are several internal diseases that may cause itch. The most common example is kidney failure. Other types of internal diseases that may cause pruritus are some types of liver disease including hepatitis C, and thyroid disease including both hyper (too much) and hypo (too little) thyroid hormone levels. Some blood disorders such as iron deficiency anemia, polycythemia vera, and multiple myeloma can cause itch. Occasionally, lymphomas may have pruritus as a component. Neurologic conditions such as pinched nerves and strokes also may lead to itch.

The treatment of itch depends on its cause. If the itch is from a skin disease such as hives or eczema, treatment of the skin disease itself generally relieves the itch. If the itch is from an internal disease, patients may require medication to be taken orally, or occasionally may receive ultraviolet light treatments to relieve the itch. Although there are many causes for pruritus, there are some basics which apply to most treatments, for example, avoiding hot bathing or showering, wearing light clothing, and a cool work or domestic environment, using mild soaps, applying moisturizing creams or lotions. Soaps often dry out the skin. Use mild soaps only in odor bearing regions. After bathing, be sure. For itchy conditions where blistering or weeping of the skin is present, such as chicken pox or poison ivy, taking a cool oatmeal bath, or using topical drying agents such as calamine, may be helpful. Although pruritus is an often disrupting and disabling symptom it generally responds well to treatment.

### DETAILED DESCRIPTION

In this invention, a combination of herbs and other natural substances is described consisting of Cactus (Opuntia ficus indica), Feng Jiao (Apis mellifera ligustica Spin) (also known as Bee Propolis), β-sitosterol, and Stearic Acid in which each of the ingredients is preferably in an amount in the range of 1.0 to 20% of the final amount of preparation. In this preparation, Soybean concentrates (containing 40% β-sitosterol used.), and Propolis dilution (5:1) is used. In the foregoing assertion, efforts have been made to find a suitable palliative and/or curative agent for the treatment of various types of skin itch.

Each component of preparation whether used in its raw form or as an extract is standardized according to its marker compound(s) and factored into final calculation of the amount of extract used for the manufacturing of final preparation. For example, if an extract is used which is 1:10 extract (such as the Cactus extract), meaning that 10 G of the component yields 1 G of extract then the required quantity of the extract for a 5% of original component will be provided by a quantity of 0.50 G extract in the preparation. In the case of soybean, a standardized 40% extract is used (Catalog # S 5753 Minimum 40% Soybean, Sigma-Aldrich, St. Louis, Mo.). In this case, a 2% final concentration of β-sitosterol is provided by 5 G of the above 40% Soybean. In the case of propolis, a 5:1 dilution in ethanol provides a 1% or original propolis by adding 5 G of the diluted propolis. Note that whereas in the case of extracts the yield is more concentrated but in the case of propolis it is a diluted form.

The extracts and dilutions corresponding to about 1-15%, are mixed together in a stainless steel tank with vacuum and heating implementation and then an amount of vegetable oil, preferably canola oil, is mixed for 10 minutes. Vacuum is then applied while heating the preparation to 40° C. to remove alcohol (such as from propolis dilution and Cactus extract), leaving a residue of alcohol not more than one percent in the final preparation. This, however, is not a critical step. The oil mixture is then filtered through muslin cloth to remove any suspended particles. Further the while the preparation is still warm, pharmaceutical grade beeswax in the amount equivalent to final preparation composition of 8%, ranging from 5-12%, is added and the mixture stirred gently for about 5 minutes at elevated temperature and then allowed to cool in appropriate containers such as laminated plastic tubes or laminated aluminum tubes or jars (laminated plastic, laminated metal or glass). The quantity of wax added is also not critical and can be adjusted to provide a consistency of ointment suitable for topical administration. Different amounts of wax can be added to produce preparations that may be useful for different purposes.

As is described in Propolis in Natural Therapeutics (1983), 2nd revised edition, published by Librairie Maloine S. A. Editeur, Paris, France, Fragrance Journal, No. 83, pp. 20-28 and pp. 36-43 (1987), and Apidologie, Vol. 22, pp. 155-162 (1991), propolis is a resin-like product, stored by bees in beehives, containing resins, beeswaxes, essential oils, pollens and flavonoids and having been used in a variety of folk medicines for a long time. It has been known that the main activities of propolis are antiseptic activity, antioxidation activity, anti-inflammatory activity, local anesthesia, virus growth-inhibitory activity, immunoregulatory activity, and macrophage activating activity, and that the main ingredients of propolis are flavonoids, aromatic carboxylic acids, and aromatic aldehydes. Honey bees collect the sap or resin from trees, such as poplar or birch, combine it with their own enzymes, and create propolis. Raw propolis is composed of 50-70% resins and balsams, 30-50% wax, 5-10% pollen and 10% essential oils. It contains Vitamin B1, 0.20 mg/100 g; vitamin B2 0.60 mg/100 g, vitamin B6 0.16 mg/g, vitamin E 3.80 mg/100 g, folic acid 7 mg/100 g, pantothenic cid 0.08 mg/100 g, inositol 6 mg/100 g. It is rich in trace minerals. And in its raw state, has 500 times more bioflavonoids than oranges. Propolis acts as a natural antibiotic, creating one of the most sterile environments in the animal kingdom has shown it offers antiseptic, antibiotic, antibacterial, antifungal, and even antiviral properties. Propolis is manufactured by collecting from the frames and boxes of the beehives and collectors place in the hives. Propolis is macerated and dissolved in pure ethyl alcohol and the mixture agitated and matured until a proportion of 20% dilution is achieved. (See: Ceschel G C, Maffei P, Sforzini A, Lombardi Borgia S, Yasin A, Ronchi C. In vitro permeation through porcine buccal mucosa of caffeic acid phenetyl ester (CAPE) from a topical mucoadhesive gel containing propolis. Fitoterapia. 2002 November;73 Suppl 1:S44-52.; Kiderman A, Torten R, Furst A L, Reinus K. Bi-lateral eosinophilic ulcers in an infant treated with propolis. J Dermatolog Treat. 2001 March;12(1):29-31.; Totan Y, Aydin E, Cekic O, Cihan Dagloglu M, Borazan M, Daglioglu K, Gultek A. Effect of caffeic acid phenethyl ester on corneal neovascularization in rats. Curr Eye Res. 2001 October;23(4):291-7; Bauer A, Geier J, Elsner P. Allergic contact dermatitis in patients with anogenital complaints. J Reprod Med. 2000 August;45(8):649-54.; Ozturk F, Kurt E, Cerci M, Emiroglu L, Inan U, Turker M, Ilker S. The effect of propolis extract in experimental chemical corneal injury. Ophthalmic Res. 2000 January-February;32(1):13-8.; Ilhan A, Koltuksuz U, Ozen S, Uz E, Ciralik H, Akyol O. The effects of caffeic acid phenethyl ester (CAPE) on spinal cord ischemia/reperfusion injury in rabbits. Eur J Cardiothorac Surg. 1999 October;16(4):458-63.; Ozturk F, Kurt E, Inan U U, Emiroglu L, Ilker S S, Sobaci G. Effect of propolis on endotoxin-induced uveitis in rabbits. Jpn J Ophthalmol. 1999 July-August;43(4):285-9.; Hepsen I F, Er H, Cekic O. Topically applied water extract of propolis to suppress corneal neovascularization in rabbits. Ophthalmic Res. 1999;31(6):426-31.; Ozturk F, Kurt E, Inan U U, Emiroglu L, Ilker S S. The effects of acetylcholine and propolis extract on corneal epithelial wound healing in rats. Cornea. 1999 July;18(4):466-71.; Gallenkemper G, Rabe E, Bauer R. Contact sensitization in chronic venous insufficiency: modern wound dressings. Contact Dermatitis. 1998 May;38(5):274-8.; Fernandez de Corres L, Diez J M, Audicana M, Garcia M, Munoz D, Fernandez E, Etxenagusia M. Photodermatitis from plant derivatives in topical and oral medicaments. Contact Dermatitis. 1996 September;35(3):184-5.; Basnet P, Matsushige K, Hase K, Kadota S, Namba T. Potent antihepatotoxic activity of dicaffeoyl quinic acids from propolis. Biol Pharm Bull. 1996 April;19(4):655-7. Huang M T, Ma W, Yen P, Xie J G, Han J, Frenkel K, Grunberger D, Conney A H. Inhibitory effects of caffeic acid phenethyl ester (CAPE) on 12-O-tetradecanoylphorbol-13-acetate-induced tumor promotion in mouse skin and the synthesis of DNA, RNA and protein in HeLa cells. Carcinogenesis. 1996 April;17(4):761-5.; Maichuk IuF, Orlovskaia L E, Andreev V P. [The use of ocular drug films of propolis in the sequelae of ophthalmic herpes] Voen Med Zh. 1995 December;(12):36-9, 80. Russian.; Magro-Filho O, de Carvalho A C. Topical effect of propolis in the repair of sulcoplasties by the modified Kazanjian technique. Cytological and clinical evaluation. J Nihon Univ Sch Dent. 1994 June;36(2):102-11.; Frenkel K, Wei H, Bhimani R, Ye J, Zadunaisky J A, Huang M T, Ferraro T, Conney A H, Grunberger D. Inhibition of tumor promoter-mediated processes in mouse skin and bovine lens by caffeic acid phenethyl ester. Cancer Res. 1993 Mar. 15;53(6):1255-61.; Magro Filho O, de Carvalho A C. Application of propolis to dental sockets and skin wounds. J Nihon Univ Sch Dent. 1990 March;32(1):4-13.; Machackova J. The incidence of allergy to propolis in 605 consecutive patients patch tested in Prague. Contact Dermatitis. 1988 April;18(4):210-2.; Frosch P J. [Current contact allergens] Z Hautkr. 1987 Dec. 1;62(23):1631-4, 1637-8. German.; Hausen B M, Wollenweber E, Senff H, Post B. Propolis allergy. (II). The sensitizing properties of 1,1-dimethylallyl caffeic acid ester. Contact Dermatitis. 1987 September;17(3):171-7.; Trevisan G, Kokelj F. Contact dermatitis from propolis: role of gastrointestinal absorption. Contact Dermatitis. 1987 January;16(1):48. No abstract available.; Savova I. [Results of treating varicose ulcer patients with propolis] Vestn Dermatol Venerol. 1987;(1):56-7; Valsecchi R, Cainelli T. Dermatitis from propolis. Contact Dermatitis. 1984 November;11(5):317. No abstract available.; Takahashi M, Matsuo I, Ohkido M. Contact dermatitis due to honeybee royal jelly. Contact Dermatitis. 1983 November;9(6):452-5.; Lutsenko S M, Pisarenko A S. [Use of water-soluble propolis for trophic ulcers of the lower extremities in arteriosclerosis obliterans] Klin Khir. 1980 July;(7):74. Russian.; Zambor M, Martanova H, Matel I. [Uses of 5% alcohol solution of propolis in the treatment of dermatomycoses (author's transl)] Cesk Dermatol. 1977 August;52(4):243-6. Slovak.).

Opuntia comprises mainly the whole plant of Opuntia ficus indica (Cactacea family) as the main constituents. Other species and varieties of Opuntia genus of the Cactaceae family are included here by reference. The reported pharmacologic properties of Opuntia include: analgesia, antiinflmmatory, antiulcerogenic, antioxidative, affecting activity of aromatase and reductase, free radical scavenger, antiviral, lowering LDL cholesterol levels, glucose-6-phosphatase and fructose-1,6-diphosphatase activity, antidiabetic, a rich source of biologically active alkaloids and other nutritional elements often considered essential for tissue growth. Optunia ficus-indica (Mission Cactus, Yellow Tuna, Prickly Pear, Indian Fig, Nopal) comprises mainly the whole plant of Opuntia ficus indica (Cactacea family) as the main constituents. It is indigenous to the Americas as well as South Asia, Southeast Asia and the Middle East. Other species and varieties of Optunia genus of the Cactaceae family are included here by reference. The reported pharmacologic properties of Optunia include: analgesia, antiinflmmatory, antiulcerogenic, antioxidative, affecting activity of aromatase and reductase, free radical scavenger, antiviral, lowering LDL cholesterol levels, glucose-6-phosphatase and fructose-1, 6-diphosphatase activity, antidiabetic, a rich source of biologically active alkaloids and other nutritional elements often considered essential for tissue growth. It is also an excellent source of beta-sitosterol. I contains mescaline, tyramine, N-methyltyramine. (See: Park E, Kahng J, Lee S H, Shin K. An anti-inflammatory principle from cactus. Fitoterapia 2001 March; 72(3):288-90., College of Pharmacy, Sookmyung Women's University, 140-742, Seoul, South Korea; Bwititi P, Musabayane C T, and Nhachi C F: Effects of Opuntia megacantha on blood glucose and kidney function in streptozotocin diabetic rats. J Ethnopharmacol 2000 March; 69 (3): 247-52; Gurrieri S, Miceli L, Lanza C M, Tomaselli F, Bonomo R P, Rizzarelli E: Chemical characterization of sicilian prickly pear (Opuntia ficus indica) and perspectives for the storage of its juice. J Agric Food Chem 2000 Nov. 20; 48 (11): 5424-5431; Mtambo M M, Mushi E J, Kinabo L D, Maeda-Machang'u A, Mwamengele G L, Yongolo M G, Temu R P: Evaluation of the efficacy of the crude extracts of Capsicum frutescens, Citrus limon and Opuntia vulgaris against Newcastle disease in domestic fowl in Tanzania. J Ethnopharmacol 1999 Dec. 15; 68 (1-3): 55-61; Loro J F, del Rio I, Perez-Santana L: Preliminary studies of analgesic and anti-inflammatory properties of Opuntia dillenii aqueous extract. J Ethnopharmacol 1999 Nov. 1; 67 (2): 213-8; Loro J F, del Rio I, Perez-Santana L: Preliminary studies of analgesic and anti-inflammatory properties of Opuntia dillenii aqueous extract.J Ethnopharmacol 1999 Nov. 1; 67 (2): 213-8; Park E H, Kahng J H, Paek E A: Studies on the pharmacological action of cactus: identification of its anti-inflammatory effect. Arch Pharm Res 1998 February; 21 (1): 30-4; Uchoa A F, Souza P A, Zarate R M, Gomes-Filho E, Campos F A: Isolation and characterization of a reserve protein from the seeds of Opuntia ficus-indica (Cactaceae). Braz J Med Biol Res 1998 June; 31 (6): 757-61; Jonas A, Rosenblat G, Krapf D, Bitterman W, Neeman I: Urol Res 1998; 26 (4): 265-70; El Kossori R L, Villaume C, El Boustani E, Sauvaire Y, Mejean: Composition of pulp, skin and seeds of prickly pears fruit (Opuntia ficus indica sp.) Plant Foods Hum Nutr 1998; 52 (3): 263-70; Ahmad A, Davies J, Randall S, Skinner G R: Antiviral properties of extract of Opuntia streptacantha. Antiviral Res 1996 May; 30 (2-3): 75-85; Trejo-Gonzalez A, Gabriel-Ortiz G, Puebla-Perez A M, Huizar-Contreras M D, Munguia-Mazariegos M R, Mejia-Arreguin S, Calva E: A purified extract from prickly pear cactus (Opuntia fuliginosa) controls experimentally induced diabetes in rats. J Ethnopharmacol 1996 December; 55 (1): 27-33; Roman-Ramos R, Flores-Saenz J L, Alarcon-Aguilar F J: Anti-hyperglycemic effect of some edible plants. J Ethnopharmacol 1995 Aug. 11; 48 (1): 25-32; Rosado J L, Diaz M: Physico-chemical properties related to gastrointestinal function of 6 sources of dietary fiber. Rev Invest Clin 1995 July-August; 47 (4): 283-9; Fernandez M L, Lin E C, Trejo A, McNamara D J: Prickly pear (Opuntia sp.) pectin alters hepatic cholesterol metabolism without affecting cholesterol absorption in guinea pigs fed a hypercholesterolemic diet. J Nutr 1994 June; 124 (6): 817-24; Fernandez M L, Lin E C, Trejo A, McNamara D J: Prickly pear (Opuntia sp.) pectin reverses low-density lipoprotein receptor suppression induced by a hypercholesterolemic diet in guinea pigs. J Nutr 1992 December; 122 (12): 2330-40; Frati A C, Xilotl Diaz N, Altamirano P, Ariza R, Lopez-Ledesma R: The effect of two sequential doses of Opuntia streptacantha upon glycemia. Arch Invest Med (Mex) 1991 July-December; 22 (3-4): 333-6; Roman-Ramos R, Flores-Saenz J L, Partida-Hernandez G, Lara-Lemus A, Alarcon-Aguilar F: Experimental study of the hypoglycemic effect of some antidiabetic plants. Arch Invest Med (Mex) 1991 January-March; 22 (1): 87-93; Fernandez M L, Trejo A, McNamara D J: Pectin isolated from prickly pear (Opuntia sp.) modifies low-density lipoprotein metabolism in cholesterol-fed guinea pigs. J Nutr 1990 November; 120 (11): 1283-90; Frati-Munari A C, Licona-Quesada R, Araiza-Andraca C R, Lopez-Ledesma R, Chavez-Negrete A: Activity of Opuntia streptacantha in healthy individuals with induced hyperglycemia. Arch Invest Med (Mex) 1990 April-June; 21 (2): 99-102; Frati-Munari A C, Altamirano-Bustamante E, Rodriguez-Barcenas N, Ariza-Andraca R, Lopez-Ledesma R: Hypoglycemic action of Opuntia streptacantha Lemaire: study using raw extracts. Arch Invest Med (Mex) 1989 October-December; 20 (4): 321-5; Frati-Munari A C, de Leon C, Ariza-Andraca R, Banales-Ham M B, Lopez-Ledesma R, Lozoya X: Effect of a dehydrated extract of nopal (Opuntia ficus indica Mill.) on blood glucose. Arch Invest Med (Mex) 1989 July-September; 20 (3): 211-6; Rodriguez-Felix A, Cantwell M: Developmental changes in composition and quality of prickly pear cactus cladodes (nopalitos). Plant Foods Hum Nutr 1988; 38 (1): 83-93; Frati Munari A C, Quiroz Lazaro J L, Altamirano Bustamante P, Banales Ham M, Islas Andrade S, Ariza Andraca C R: The effect of various doses of nopal (Opuntia streptacantha Lemaire) on the glucose tolerance test in healthy individuals. Arch Invest Med (Mex) 1988 April-June; 19 (2): 143-8; Frati-Munari A C, Gordillo B E, Altamirano P, Ariza C R: Hypoglycemic effect of Opuntia streptacantha Lemaire in NIDDM. Diabetes Care 1988 January; 11 (1): 63-6; Frati-Munari A C, Yever-Garces A, Islas-Andrade S, Ariza-Andraca C R, Chavez-Negrete A: Studies on the mechanism of “hypoglycemic” effect of nopal (Opuntia sp.). Arch Invest Med (Mex) 1987 January-March; 18 (1): 7-12; Ma W W, Jiang X Y, Cooks R G, McLaughlin J L, Gibson A C, Zeylemaker F, Ostolaza C N: J Nat Prod 1986 July-August; 49 (4): 735-7; Ibanez-Camacho R, Meckes-Lozoya M: Effect of a semipurified product obtained from Opuntia streptacantha L. (a cactus) on glycemia and triglyceridemia of rabbit. Arch Invest Med (Mex) 1983 October-December; 14 (4): 437-43; Frati-Munari A C, Fernandez-Harp J A, de la Riva H, Ariza-Andraca R, del Carmen Torres M: Effects of nopal (Opuntia sp.) on serum lipids, glycemia and body weight. Arch Invest Med (Mex) 1983 April-June; 14 (2): 117-25; Ibanez-Camacho R, Meckes-Lozoya M, Mellado-Campos V: The hypoglucemic effect of Opuntia streptacantha studied in different animal experimental models. J Ethnopharmacol 1983 March; 7 (2): 175-81; Ibanez-Camacho R, Roman-Ramos R: Hypoglycemic effect of Opuntia cactus. Arch Invest Med (Mex) 1979; 10 (4): 223-30; Woodard R W, Craig J C, Bruhn J G: Acta Chem Scand B 1978; B32 (8): 619-20; The absolute configuration of the cactus alkaloid (−)-calipamine. Mukerji S K, Ting I P: Malic dehydrogenase isoenzymes in green stem tissue of Opuntia: isolation and characterization. Arch Biochem Biophys 1969 May; 131 (2): 336-51; Mukerji S K, Ting I P: Malate dehydrogenase (decarboxylating) (NADP) isoenzymes of Opuntia stem tissue. Mitochondrial, chloroplast, and soluble forms. Biochim Biophys Acta 1968 Oct. 8; 167 (2): 239-49; Satta M A, Sisini A: Glucose-6-phosphatase and fructose-1,6-diphosphatase activity in Opuntia ficus indica. Boll Soc ltal Biol Sper 1964 Sep. 30; 40 (18): 1109-10.) Soybean extract containing a minimum of 40% β-sitosterol as used in this invention (Sigma Chemicals Catalog S5753), which also contains campesterol, dihy dro-brassicacasterol prepared according to the method of N. Kozumi, et al., Chem. Pharm. Bull., 27:38, 1979. The source of β-sitosterol however is not relevant. It could be obtained from natural sources or from synthetic sources. β-sitosterol (C29H50O, molecular weight 414.72) is a common sterol in plants. It is generally isolated from wheat germ, soybean or corn oil. Sterols are important cyclized triterpenoids that perform many critical functions in cells. Phytosterols such as campesterol, stigmasterol and β-sitosterol in plants, ergosterol in fungi and cholesterol in animals are each primary components of the cellular and sub-cellular membranes in their respective cell types. The dietary source of phytosterols in humans comes from vegetables and plant oils. The estimated daily phytosterol content in the conventional western-type diet is approximately 250 mg in contrast to a vegetable diet, which would provide double that amount. Although having no nutritional value to humans, phytosterols have recently received a great deal of attention due to their possible anti-cancer properties and their ability to decrease cholesterol levels when fed to a number of mammalian species, including humans. Phytosterols aid in limiting cholesterol absorption, enhance biliary cholesterol excretion and shift cholesterol from atherosclerotic plaque. While many of the mechanisms of action remain unknown, the relationship between cholesterol and phytosterols is apparent. This is perhaps not surprising given that chemically, phytosterols closely resemble cholesterol in structure. The major phytosterols are β-sitosterol, campesterol and stigmasterol. Others include stigmastanol (β-sitostanol), sitostanol, desmosterol, chalinasterol, poriferasterol, clionasterol and brassicasterol. (Gould R. G., Jones R. J., LeRoyu G. V., Wissler R. W., Taylor C. B.; Absorbability of B-sitosterol in humans; Metabolism, (August) 1969; 18(8): 652-662. Tabata T., Tanaka M., Lio T.; Hypocholesterolemic activity of phytosterol. II; Yakugaku Zasshi, 1980; 100(5): 546-552. Hepistall R. H., Porter K. A.; The effect of β-sitosterol on cholesterol-induced atheroma in rabbits with high blood pressure; Br. J. Experimental Pathology, 1957; 38: 49-54.). The role of phytosterols, particularly, β-sitosterol in stimulating human stem cells and particularly promoting hair growth has not been reported yet. Several novel applications of phytosterols including β-sitosterol have been reported. The U.S. Pat. No. 5,965,449 to Novak describes a method of assessing risk for cardiovascular disease and other disorders and phytosterol-based compositions useful in preventing and treating cardiovascular disease and other disorders. The level of serum campesterol and β-sitosterol are determined and their ratio is correlated with the risk of cardiovascular or a related disorder. The U.S. Pat. No. 5,523,087 to Shlyankevich is for a pharmaceutical composition for the treatment of diabetic male sexual dysfunction; it contains physosterogens, phosphatidyl choline, β-sitosterol, Damiana leaf extract and vitamins and minerals. The U.S. Pat. No. 5,486,510 to Bouic, et al., is for a mixture of β-sitosterol glucoside and β-sitosterol is administered to persons for the modulation or control of immune responses. The U.S. Pat. No. 5,747,464 to See is for a composition for inhibiting absorption of fat and cholesterol from the gut and a method for making and using the composition. The composition comprises β-sitosterol bound irreversibly to pectin to form a β-sitosterol and pectin complex. The U.S. Pat. No. 5,118,671 to Bombardelli, et al., is for complexes formed between aescin, cholesterol or β-sitosterol and phospholipids and a method for producing an anti-inflammatory effect is also described.

Olive Oil is a complex compound made of fatty acids, vitamins, volatile components, water-soluble components and microscopic bits of olive. Primary fatty acids are Oleic and linoleic acid. Oleic acid is monosaturated and makes up 55-85% of olive oil. Linoleic is polyunsaturated and makes up about 9%. Linolenic, which is polyunsaturated, makes up 0-1.5%. Vitamins are Vitamin E and carotene. The levels of these acids (actually bound to glycerol as triglycerides) varies during the different maturation stages of the olive, varies with the variety and the growing conditions. It is generally accepted that cooler areas (e.g., Tuscany) will give oil with higher oleic acid than warmer climates. Most of the research however has be reported as individual studies so that comparisons are like apples and oranges. However there is a major trial underway in all the major Mediterranean Producing countries with some 10 or so varieties. This study will give more definitive information. Regarding the poly-unsaturated fatty acids (PUFAs) there is a wide range acceptable for EVO, however the Linolenic has to be less than 0.9%. There is no problem if the levels are higher e.g., 1.5% regarding the olive oils nutritional value. But the Linolenic acid level is used to establish the authenticity of the olive oil. Seed oils like Canola have higher levels of Linolenic acid. Also the higher the level of unsaturation ie more PUFAs leads to a less stable oil, however this has to be counterbalanced by the levels of antioxidants that protect the oil (these will also vary by similar factors to the fatty acid profile as well as stress e.g., drought. A higher Linolenic than the IOOC may actually be of benefit nutritionally for reasons other than those associated with oleic acid.

Other constituants: Phenols, free fatty acids, peroxide, triacylglycerols (TAG), diacylglycerols (DAG), and monoacylglycerols (MAG), thiobarbituric acid reactive substances (TBARS), Pheophytin A andand many other substances make up olive oil. The flavenoid polyphenols in olive oil are natural anti-oxidants, which have been shown to have a host of beneficial effects from healing sunburn to lowering cholesterol, blood pressure, and risk of coronary disease. There are as many as 5 mg of antioxidant polyphenols in every 10 grams of olive oil. Many other nut and seed oils have no polyphenols. (See also: Essential fatty acid deficiency in renal failure: can supplements really help? J Am Diet Assoc. 1997 October;97(10 Suppl 2):S150-3; Peck L W, Monsen E R, Ahmad S. Effect of three sources of long-chain fatty acids on the plasma fatty acid profile, plasma prostaglandin E2 oncentrations, and pruritus symptoms in hemodialysis patients. Am J Clin Nutr. 1996 August;64(2):210-4; Benitez del Castillo J M, del Aguila C, Duran S, Hernandez J, Garcia Sanchez J. Influence of topically applied cyclosporine A in olive oil on corneal epithelium permeability. Cornea. 1994 March; 13(2): 136-40; Bond R, Lloyd D H. A double-blind comparison of olive oil and a combination of evening primrose oil and fish oil in the management of canine atopy. (Vet Rec. 1992 Dec. 12;131(24):558-60.)

Natural beeswax contains many still yet unidentified components that have for centuries provided healing properties for many ailments. Propolis is just one such example. The isolation of D-002 series of alcohols provides the clue to its anti-inflammatory activity; flavolins and other minor components provide impart many pharmacologic properties to beeswax. (See: Carbajal D, Molina V, Valdes S, Arruzazabala M L, Mas R, Magraner J: Anti-inflammatory activity of D-002: an active product isolated from beeswax. Prostaglandins Leukot Essent Fatty Acids 1998 October; 59 (4): 235-8.; Tsigouri A, Menkissoglu-Spiroudi U, Thrasyvoulou A T, Diamantidis G C: Determination of fluvalinate residues in beeswax by gas chromatography with electron-capture detection. J AOAC Int 2000 September-October;83(5):1225-8; Carbajal D, Molina V, Noa M, Valdes S, Arruzazabala M L, Aguilar C, Mas R: Effect of D-002 on gastric mucus composition in ethanol-induced ulcer. Pharmacol Res 2000 October;42(4):329-32; Aichholz R, Lorbeer E: Investigation of combwax of honeybees with high-temperature gas chromatography and high-temperature gas chromatography-chemical ionization mass spectrometry. II: High-temperature gas chromatography-chemical ionization mass spectrometry. J Chromatogr A 2000 Jun. 23;883(1-2):75-88; Noa M, Carbajal D, Molina V, Valdes S, Mas R: Comparative study of D-002 versus sulfasalazine on acetic acid-induced colitis in rats. Drugs Exp Clin Res 2000;26(1):13-7; Noa M, Mas R, Carbajal D, Valdes S: Effect of D-002 on acetic acid-induced colitis in rats at single and repeated doses. Pharmacol Res 2000 April;41(4):391-5; Aichholz R, Lorbeer E: Investigation of combwax of honeybees with high-temperature gas chromatography and high-temperature gas chromatography-chemical ionization mass spectrometry. I. High-temperature gas chromatography. J Chromatogr A 1999 Sep. 10;855(2):601-15; Turi M S, Matray E S: Determination of acrinathrin residues in honey and beeswax. Acta Vet Hung 1999;47(2):173-9; Rodriguez M D, Gamez R, Sanchez M, Garcia H: Developmental toxicity of D-002 (a mixture of aliphatic primary alcohols) in rats and rabbits. J Appl Toxicol 1998 September-October; 18 (5): 313-6; Carbajal D, Molina V, Valdes S, Arruzazabala L, Mas R: Anti-ulcer activity of higher primary alcohols of beeswax. J Pharm Pharmacol 1995 September; 47 (9): 731-3; Carbajal D, Molina V, Valdes S, Arruzazabala M L, Mas R, Magraner J: Anti-inflammatory activity of D-002: an active product isolated from beeswax. Prostaglandins Leukot Essent Fatty Acids 1998 October;59(4):235-8.; Noa M, Mas R: Effect of D-002 on the pre-ulcerative phase of carrageenan-induced colonic ulceration in the guinea pig. J Pharm Pharmacol 1998 May;50(5):549-53.; Rodriguez M D, Gamez R, Sanchez M, Garcia H: Developmental toxicity of D-002 (a mixture of aliphatic primary alcohols) in rats and rabbits. J Appl Toxicol 1998 September-October;18(5):313-6; Liu F, Sun D: Active constituents lowering blood-lipid in beeswax. Zhongguo Zhong Yao Za Zhi 1996 September;21(9):553-4, 576; Carbajal D, Molina V, Valdes S, Arruzazabala L, Rodeiro I, Mas R, Magraner J: Possible cytoprotective mechanism in rats of D-002, an antiulcerogenic product isolated from beeswax. J Pharm Pharmacol 1996 August;48(8):858-60.; Dorset D L: The crystal structure of waxes. Acta Crystallogr B 1995 Dec. 1;51 (Pt 6):1021-8; Carbajal D, Molina V, Valdes S, Arruzazabala L, Mas R: Anti-ulcer activity of higher primary alcohols of beeswax. J Pharm Pharmacol 1995 September;47(9):731-3; Zanoschi C, Ciobanu C, Verbuta A, Frincu D: The efficiency of some natural drugs in the treatment of burns. Rev Med Chir Soc Med Nat lasi 1991 January-June;95(1-2):63-5; Kuenzi M J, Connolly B A, Sherwood O D: Relaxin acts directly on rat mammary nipples to stimulate their growth. Endocrinology 1995 Ju1;136(7):2943-7; Li M, Nelson D L, Sporns P: Determination of menthol in honey by gas chromatography. J AOAC Int 1993 November-December;76(6):1289-95; Ludianskii E A: The use of the products of bee raising in medicine. Feldsher Akush 1989 September;54(9):36-9; Blum M S, Jones T H, Rinderer T E, Sylvester H A: Oxygenated compounds in beeswax: identification and possible significance. Comp Biochem Physiol B 1988;91(3):581-3; Chlorazak T, Szaflarski J, Seferowicz E, Scheller S: Preliminary evaluation of clinical usefulness of propolis (beeswax) preparations. Przegl Lek 1971;2 8(12):828-31.

[Composition of a Preferred Embodiment]

| Ingredient | Percentage |
|---|---|
| Propolis | 1 |
| Opuntia | 5 |
| β-sitosterol | 2 |
| Stearic Acid | 3 |
| Olive Oil | to 100 |
| Alcohol | qs |

The preparation described above was applied to itching surface in different situations, chemical itch, wound-healing itch, anal itch, vaginal itch, irritant itch, nervous itch and in all instances an immediate relief was observed. It will be appreciated by those skilled in this art that the above approximate weight percentages are dependent on generally expected potencies of the components, whereby the relative weight percentages will vary sometimes substantially from the above individual amounts. It will be within the skilled person's knowledge with this disclosure that the objects of the present invention require the inclusion of each of the components in relative approximate weight percentages above.

1. A composition for topical application to a body surface to treat itch in humans and animals comprising of Bee Propolis, 0.10-10%; Stearic Acid, 1-10%,; Opuntia Ficus-indica, 1-10%; β-sitosterol, 1-10%; Natural Beeswax, 2-20%; Vegetable Oil QS to Volume.

2. The composition of claim 1 wherein the said composition contains Propolis and Opuntia as alcoholic extracts.

3. The composition of claim 1 wherein the said composition contains β-sitosterol and stearic acid derived from either natural or synthetic source.

4. The composition of claim 1 wherein the said vegetable oil is olive oil.

5. The composition of claim 1 wherein the said composition is used for the treatment of skin itch in humans and animals including scratch reaction itch, anal itch, vaginal itch, scalp itch or irritant itch due to plants (e.g., poison ivy), insect bite, sunburn, chemical itch, eczema, pruritis dermatitis, diabetic itch, aging skin itch, athletes foot itch, chickenpox, jock itch, hives, itch of healing burns and wounds, dry winter skin itch, and stress-related scalp itchc.

6. The composition of claim 1 wherein the said composition is an ointment, cream, lotion, liquid, aerosol, powder, poultice, dressing, spray or a dosage form suitable for topical administration or applicationh and containing ingredients suitable to manufacture these chosen dosage forms.
